(19) United States
(12) Patent Application Publication
(10) Pub. No.: US 2021/0304859 A1
(43) Pub. Date: Sep. 30, 2021

(54) CLOUD-BASED MEDICAL RECORD MANAGEMENT SYSTEM WITH PATIENT CONTROL

(71) Applicant: Nariman Bharucha, Kingston/Saint Andrew (JM)

(72) Inventor: Nariman Bharucha, Kingston/Saint Andrew (JM)

(21) Appl. No.: 17/215,972

(22) Filed: Mar. 29, 2021

Related U.S. Application Data
(60) Provisional application No. 63/000,909, filed on Mar. 27, 2020.

(51) Int. Cl. G16H 10/65; G16H 20/10; G06F 21/34; G06F 21/62

(52) U.S. Cl. CPC G16H 10/65; G06F 21/6245; G06F 21/34; G16H 20/10

(57) ABSTRACT

A cloud-based medical record management system providing patient control for storing, accessing, managing and sharing their patient medical records. The patient is provided complete control over access to his medical records and is provided a transparent system for sharing and transmitting to various medical providers over various medical system platforms. In this way, medical professional, medical facilities and other healthcare-related organizations are able to communicate and share medical records, under patient specific control, in a rapid way and in real-time.

### CROSS REFERENCE TO RELATED APPLICATIONS

This application claims the benefit of U.S. Provisional Application 63/000,909, filed Mar. 27, 2020, which is incorporated herein in its entirety.

### FIELD OF THE INVENTION

The present invention relates generally to medical record management, and more particularly, to a cloud-based medical record management system for providing patient control over storing, managing, securing and controlling patient medical records.

### BACKGROUND OF THE INVENTION

Medical records related to a patient's health information are critical to the provision of medical care by health care providers. Personal and medical information must be accessible and manageable by medical professionals in order to provide effective patient medical care. Hospitals, medical professional practices and individual doctor's offices are charged with the collection, updating and maintenance of such personal and medical information during each patient encounter where medical treatment is sought and delivered. Patient medical records are typically created, accessed and updated by doctors, nurse practitioners, nurses and other medical personnel during various stages of patient treatment. Given the diversification and specialization in the medical field a patient may be treated by multiple doctors or specialists and may receive treatments in multiple medical facilities. In addition, a patient may require unexpected emergency care in a hospital emergency room, for example, that will require access to patient medical records and also generate additional updates from this medical facility that the patient may be unfamiliar. Of course, it is not uncommon for there to be required access to such personal and medical information by medical professionals outside of a particular patient's hospital or general practitioner's office in seeking outside expert opinions and/or evaluation of medical imaging and laboratory test results.

Traditionally, such medical records were paper-based in format and required significant amounts of physical storage. Furthermore, the paper records may have been stored in multiple locations making their aggregation difficult, time consuming and costly Security of paper records is also a major issue in modern medical care scenarios. In view of these issues, the emergence and use of electronic health records (EHR) (also sometimes referred to as electronic medical records (EMR)) has grown exponentially and is replacing the use of paper records in their entirety.

EMRs, a digital version containing the same or substantially the same information as a paper medical record, offer medical professionals the power and convenience of accessing and managing an enormous amount of patient data in ways simply not practical in a paper-based system. EMRs are structured to contain a patient's complete medical history with medical information including, but not limited to, medical history, prescription history, allergies, vital signs, immunization history, laboratory testing results, medical imaging results, personal statistics (e.g., height, weight, eye color, gender, etc.), emergency contacts, family medical histories and medical insurance coverage information.

In addition to these many advantages over paper-based record, EMRs also provide enhanced security and privacy protection. In the United States, the medical field is charged with compliance to all current health information laws and regulations. One major U.S. federal regulation is the Health Insurance Portability and Accountability Act (HIPAA) that regulates the use and disclosure of defined protected health information. In particular, these regulations provide restrictions on disclosure and access to protected health information to and by third parties. HIPAA may require any access to hardware or other equipment containing medical information be carefully monitored and controlled with access limited to authorized personnel only.

HIPAA is also concerned with ensuring data integrity and that data stored in a medical records system cannot be accessed or altered in any unauthorized way. The so-called HIPAA Security Rule provides for additional constraints with respect to electronic data security and the transmission and storage of protected health information. Additionally, the large amounts of data that needs electronic storage in today's medical environments present unique challenges to the medical providers and information infrastructure providers in terms of infrastructure requirements to effectively manage the data. Significantly, a HIPAA violation may result in a federal investigation with possible civil penalty money damages. Therefore, compliance is an area taken very seriously by the medical professional ecosystem.

Not unexpectedly with the establishment of HIPAA and today's focus on personal data security including, but not limited to, personal medical health information there is an ever-increasing demand for patient control and protection of their personal medical health information. For example, patients are currently provided portal access by a medical provider that allows the patient to access their electronic medical records, as maintained by the particular medical provider, through a login process to the provider's patient portal. However, the patient portal is specific to that provider's system and associated medical data and is not typically in communication with other systems that may be required to treat the patient and/or that hold other patient records necessary for treatment. As such, patients continue to demand better control of and access to their medical histories with higher degrees of transparency, security and portability across multiple medical providers.

Accordingly, there is need for a solution providing improved patient control and access to their medical histories with higher degrees of transparency, security and portability across multiple medical providers.

### SUMMARY OF THE INVENTION

The present invention is directed to a cloud-based medical record management system that provides patient control for storing, accessing, managing, securing and sharing their patient medical records.

In a first implementation of the invention, a cloud-based medical record management system is provided employing a HIPAA-compliant cloud comprising at least one or more servers and databases. The HIPAA-compliant cloud facilitates the storage of patient medical records that are under patient-control thereby allowing the patient to directly control and provide access to such medical-related information by and through two primary mechanisms, in particular, a patient device for executing mobile applications and a control card. The HIPAA-compliant cloud facilitates communications between a variety of medical facilities and health care establishments including but not limited to hospitals, clinics, emergency services, pharmacies and telemedicine providers. Electronic communications between various networks, the HIPAA-compliant cloud and the aforementioned institutions are facilitated by communications links.

In a second aspect, a method is provided for patient control for storing, accessing, managing, securing and sharing their patient medical records using the cloud-based medical system.

In a third aspect, a healthcare information exchange system is provided comprising one or more servers and one or more databases, the healthcare information exchange system being in communication with a plurality of healthcare entities over one or more networks, and the one or more databases storing a plurality of patient specific medical records, a patient device comprising a processor and a memory storing instructions that when executed cause the processor to perform operations comprising transmitting, under control by a patient associated with the patient device, a patient medical record access request specific to the patient, the patient medical access request including at least a patient identification code and an access code that are specific to identifying and authenticating the patient. Wherein the healthcare information exchange system, in response to receiving the patient medical record access request through the one or more servers, verifies an authenticity of the patient using the patient identification code and the access code and, only if the authenticity is verified, retrieves, from the one or more databases, a particular one patient specific medical record of the plurality of patient specific medical records, the particular one patient specific medical record being associated with the patient, and transmits, over a particular one network of the one or more networks, the at least one patient specific medical record to a particular one healthcare entity of the plurality of healthcare entities.

In a fourth aspect, patient control over and access to personal medical records may be facilitated by a patient device comprising a mobile application that when executed delivers operations for patient control for storing, accessing, managing and sharing their patient medical records.

In a fifth aspect, patient control over and access to personal medical records may be facilitated by a patient control card comprising a processor, memory power supply, magnetic strip and smart card interface. When the patient control card is in physical possession of the patient and presented by the patient this will facilitate a method for patient control for storing, accessing, managing, securing and sharing their patient medical records.

In another aspect, the patient control delivered the patient device or patient control card, as the case may be, facilitates procuring necessary prescription drugs in view of the patient's direct control and authority over fulfillment in view of the communicated price levels and visibility into the price variations among the competing pharmacies.

In another aspect, the patient device may be a mobile device such as a smartphone, laptop computer, tablet and/or wearable device.

In another aspect, the patient control card may include a patient photograph, provider name, patient account number, patient name and logo.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a high-level block diagram of a cloud-based medical record management system in accordance with a first embodiment of the invention.

FIG. 2 presents an illustrative patient device configured for use with the cloud-based medical record management system of FIG. 1 in accordance with an embodiment.

FIG. 3 presents a front view of an illustrative patient control card for use with the cloud-based medical record management system of FIG. 1 in accordance with an embodiment.

FIG. 4 presents a back view the illustrative patient control card of FIG. 3 for use with the cloud-based medical record management system of FIG. 1 in accordance with an embodiment.

FIG. 5 presents a high-level functional block diagram of the illustrative patient control card of FIGS. 3 and 4.

FIG. 6 presents an illustrative patient medical record affiliated with a patient using the cloud-based medical record management system of FIG. 1 in accordance with an embodiment.

FIGS. 7 and 7A presents a flowchart of illustrative operations for patient control for storing, accessing, managing, securing and sharing their patient medical records using the cloud-based medical record management system of FIG. 1 in accordance with an embodiment.

FIG. 8 presents a flowchart of illustrative operations for new patient onboarding using the cloud-based medical record management system of FIG. 1 in accordance with an embodiment.

FIG. 9 presents a flowchart of illustrative operations for prescription fulfillment using the cloud-based medical record management system of FIG. 1 in accordance with an embodiment.

FIG. 10 presents a high-level block diagram of an exemplary computer for executing the operations shown in FIGS. 7-9 in accordance with an embodiment.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in the Figures herein. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a cloud-based medical record management system that provides patient control for storing, accessing, managing and sharing their patient medical records in real-time.

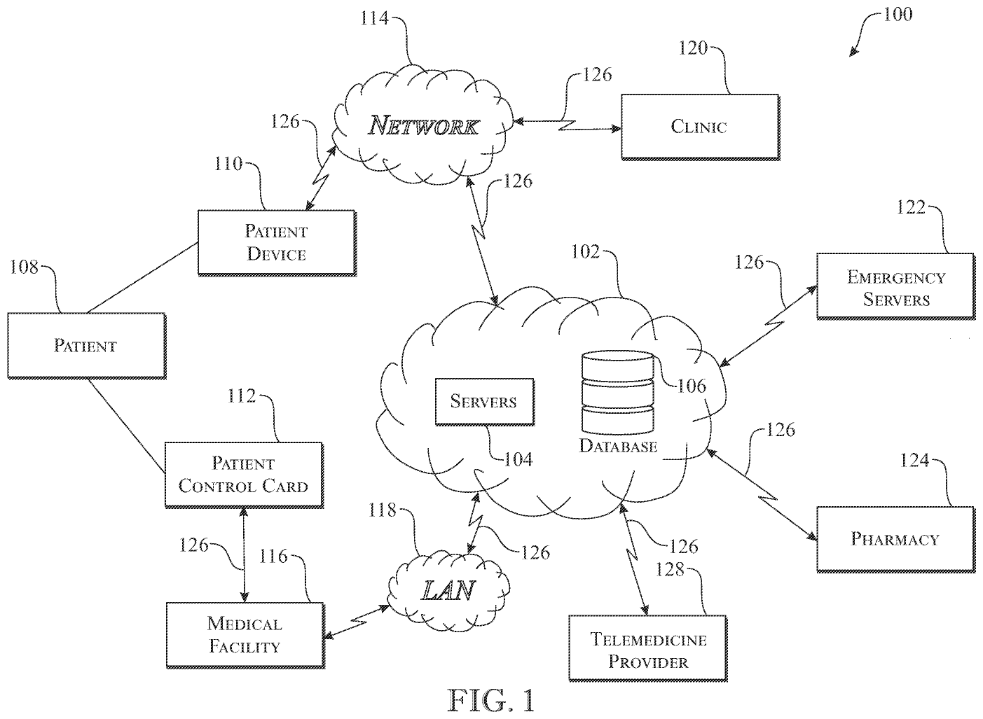

FIG. 1 presents a high-level block diagram of a cloud-based medical record management system 100 in accordance with a first embodiment of the invention. As shown for instance in FIG. 1, the cloud-based medical record management system 100 includes a healthcare information exchange system 102 (alternatively referred to herein as a HIPAA-compliant cloud) comprising at least servers 104 and databases 106. As will be detailed herein below, the healthcare information exchange system 102 in accordance with the embodiment facilitates the storage of patient medical records that are under direct patient-control (e.g., patient 108) thereby allowing the patient 108 to control and provide access to such medical-related information. As shown, the patient 108 employs two primary mechanisms to facilitate such control, in particular, patient device 110 and/or patient control card 112.

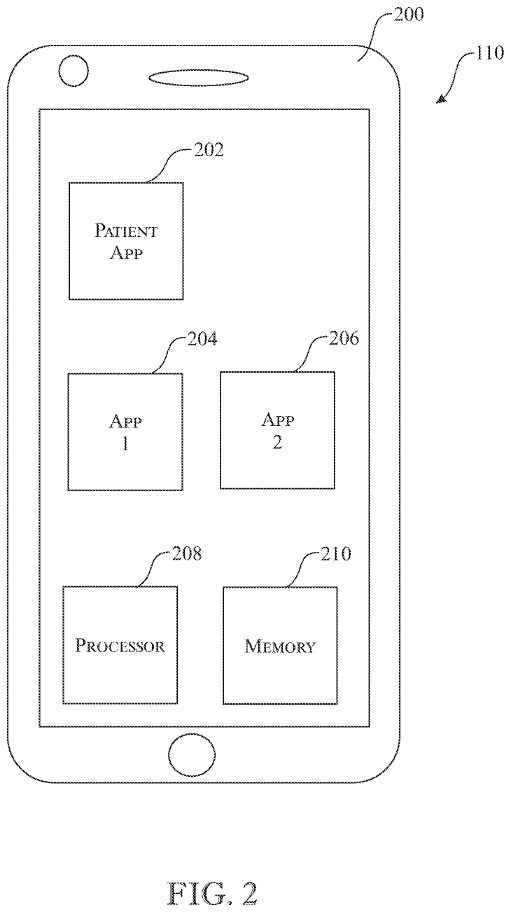

Turning our attention briefly to FIG. 2, an illustrative patient device 110 configured as mobile device 200 is shown for deployment with the cloud-based medical record management system of FIG. 1 in accordance with an embodiment. The mobile device 200 includes processor 208, memory 210, patient application (app) 202, application “1” 204 and application “2” 206 that are configured in a well-known fashion as mobile application programs. Upon launching and executing the patient app 202 the patient is in direct and total control of access to their individual medical records. As will be appreciated, while the mobile device 200 is configured as a smartphone any number of mobile of devices that execute mobile applications may be utilized with the principles of the disclosed embodiments herein. As such, a “mobile device” in the context herein may comprise a wide variety of devices such as smartphones, laptop computers, tablets, and wearable device, to name just a few. The operations of the patient app 202 will be discussed in much greater detail herein below for understanding the delivery of patient-controlled medical record access and control in the cloud-based medical record management system 100.

As shown in the cloud-based medical record management system 100, the patient 108 may alternatively utilize patient control card 112 for the delivery of patient-controlled medical record access and control in the cloud-based medical record management system 100. In accordance with an embodiment, the patient 108 will present the patient control card when seeking medical attention from one of a plurality of healthcare entities, for example, medical facility 116. The patient control card 112 when presented and swiped in a well-known manner using a card reader at the medical facility 116 will trigger a communication, over local area network (LAN) 118 from the medical facility 116 across the communications links 126 to the HIPAA-compliant cloud 102 where medical records of the patient 108 are stored. In this way, the patient is in direct and total control of access to their individual medical records on a real-time basis.

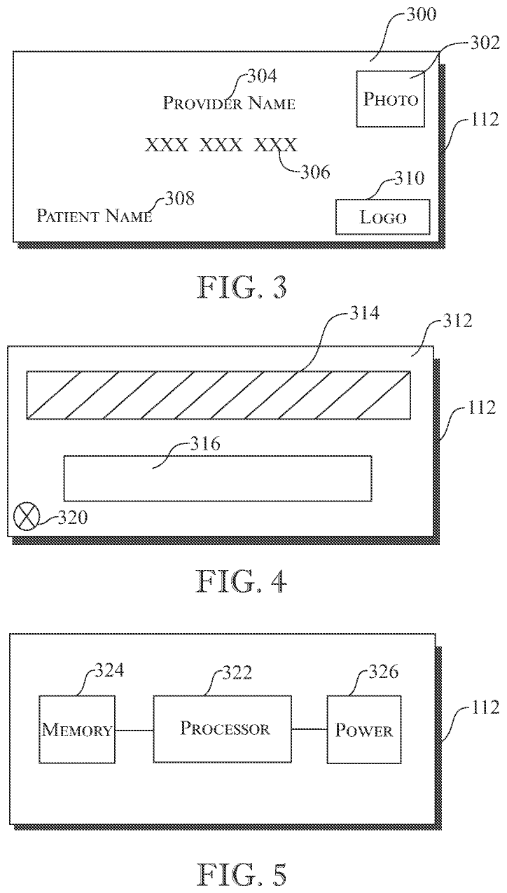

Turing our attention to FIGS. 3-5, FIG. 3 presents a front view 300 of the patient control card 112 for deployment with the cloud-based medical record management system 100 of FIG. 1 in accordance with an embodiment. As shown, the patient control card 112 is the size and shape of a conventional credit card with patient photograph 302, provider name 304, patient account number 306, patient name 308 and logo 310. Referring to FIG. 4, a back view 312 of the patient control card 112 of FIG. 3 is shown comprising magnetic strip 314 coded with card specific information in a well-known fashion and patient signature block 316. Smart card interface 320 is further provided for delivery of smart card capabilities to the patient control card in a well-known fashion and, as will be appreciated, the smart card interface 320 may also be located on the front of the patient control card instead of the back as shown in FIG. 4.

Further, FIG. 5. presents a high-level functional block diagram of the patient control card 112 of FIGS. 3 and 4. As shown, the patient control card 112 comprises processor 322, memory 324 and power supply 326. As will be appreciated, there exist well-known standards governing the design of smart cards such as the patient control card 112. For example, international standard ISO 7816 is one such standard and smart card interface and contactless card standards include ISO 14443 and 15693. The illustrative embodiments herein may include either well-understood contact-based or contactless (e.g., RF) based designs for the patient control card 112. As with the patient app 202 detailed above, the operations of the patient control card 112 will be discussed in much greater detail herein below for understanding the delivery of patient-controlled medical record access and control in a the cloud-based medical record management system 100.

Turning our attention back to FIG. 1, in an embodiment, upon launching and executing patient app 202 the patient 108 is in direct and total control of access to their individual medical records. In this way, the patient 108 may share his medical records by and through the HIPAA-compliant cloud 102 with a plurality of medical facilities and health care entities including but not limited to medical facility 116 (e.g., a hospital), clinic 120 (e.g., a ready care facility), emergency services 122 (e.g., an emergency room), pharmacy 124 and telemedicine provider 128. Electronic communications between the network 114, the HIPAA-compliant cloud 102, the medical facility 116, the clinic 120, the emergency services 122, the pharmacy 124, the telemedicine provider 128, the patient device 110 and the patient 108 are facilitated by communications links 126 in accordance with any number of well-known communications protocols and methods (e.g., wireless communications).

As shown, the HIPAA-compliant cloud 102 comprises at least servers 104 and databases 106. Cloud, cloud service, cloud server and cloud database are broad terms and are to be given their ordinary and customary meaning to one of ordinary skill in the art and includes, without limitation, any medical database, data repository or storage media which store medical information typically associated with and managed by patients, doctors, nurses, technicians, lab centers, hospitals, medical imaging facilities and health insurance companies, to name just a few. Medical information is a broad term and is to be given its customary meaning to a person of ordinary skill in the art and includes, without limitation, exams, studies, diagnoses, lab results, test results, images, medical history, treatment history, prescription history, payment information, among other types of like information. Similarly, personal information is a broad term and is to be given its ordinary and customary meaning to a person of ordinary skill in the art and includes, without limitation, physical addresses, email addresses, social security numbers, credit card numbers, bank accounts, medical billing, medical insurance policies, any HIPAA-related releases and other types of like information relating to a particular person or patient.

A cloud service may include one or more cloud servers and cloud databases that provides for the remote storage of medical information as hosted by a third-party service provider or operator. A cloud server may include an HTTP/HPTTPS server sending and receiving messages in order to provide web-browsing interfaces to client web browsers as well as web services to send data to integrate with other interfaces (e.g., as executed on the patient device 110). The cloud server may be implemented in one or more well-known servers and may send and receive medical records, medical information, patient supplied information and profile/configuration data that may be transferred to, read from or stored in a cloud database (e.g., databases 106). A cloud database may include one or more physical servers, databases or storage devices as dictated by the cloud service's storage requirements. The cloud database may further include one or more well-known databases (e.g., an SQL database) or a fixed content storage system to store medical information, profile information, configuration information or administration information as necessary to execute the cloud service. In various embodiments, one or more networks providing computing infrastructure on behalf of one or more patients may be referred to as a cloud, and resources may include, without limitation, data center resources, applications (e.g., software-as-a-service or platform-as-a-service) and management tools. In this way, in accordance with various embodiments, the patients may control and provide access to their medical records in a fully transparent fashion without any required understanding of the underlying hardware and software necessary to interface, communicate, manipulate and exchange such medical records.

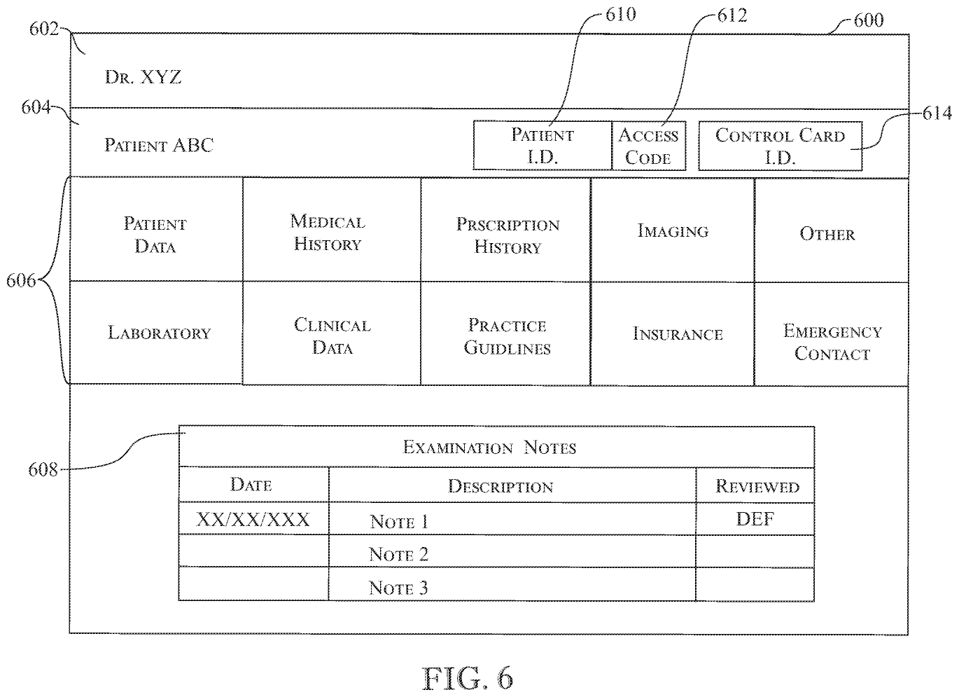

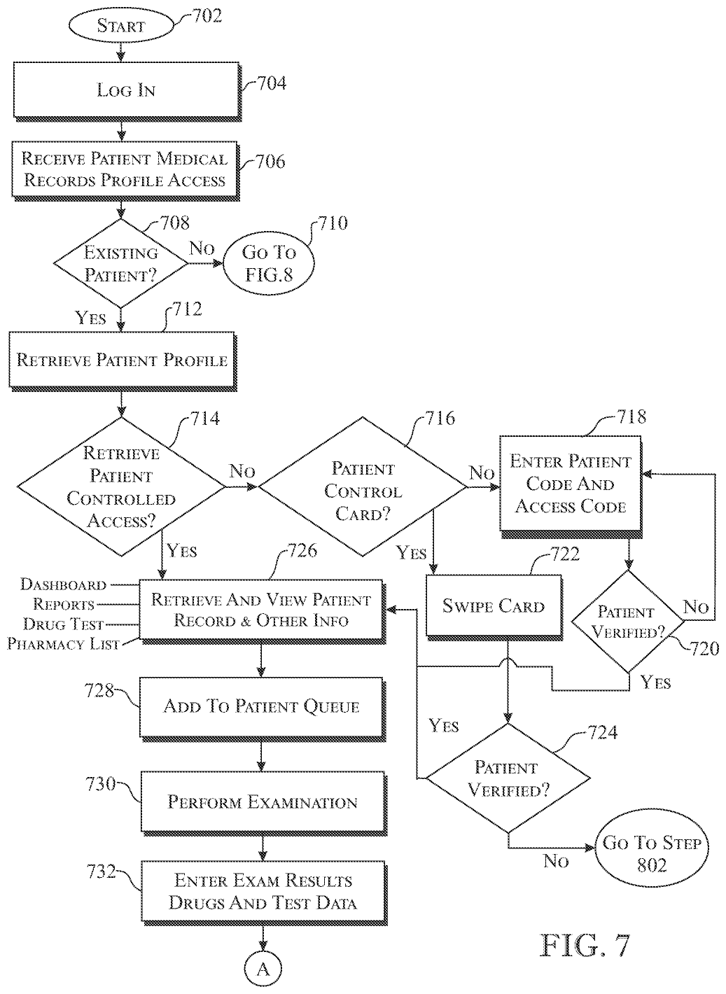

Turning our attention to FIG. 6, an illustrative patient medical record 600 is shown as affiliated with a patient (e.g., the patient 108) using the cloud-based medical record management system of FIG. 1 in accordance with an embodiment. Illustratively, the medical record 600 comprises (i) medical provider field 602 for storing information specific to the medical provider (e.g., doctor name, medical facility, etc.), (ii) patient information field 604 for storing information specific to a patient (e.g., patient name, patient ID 610, access code 612 and control card ID 614), (iii) medical information fields 606 for holding medical information specific to the patient (e.g., patient 108) such as patient data, medical history, prescription history, imaging results, laboratory results, clinical data, practice guidelines, insurance information, emergency contacts and other information, and (iv) examination field 608 for storing examination results specific to the patient 108.

In accordance with an embodiment, the patient 108 using the patient device 110 makes a request and/or grants access to his medical information for examination purposes as an instantiation of patient medical record 600 as stored in databases 106. This request or access grant is made, illustratively, by launching the patient app 202 in a well-understood manner that establishes connectivity with the HIPAA-compliant cloud 102 through the network 114 (e.g., the Internet) over communication links 126. Network 114 may be any type or form of network and may include, without limitation, a point-to-point network, a broadcast network, a wide area network, a local area network, a telecommunications network, a data communications network, a computer network, an asynchronous transfer mode (ATM) network, a synchronous optical network (SONET), a wire-line network and/or wireless network.

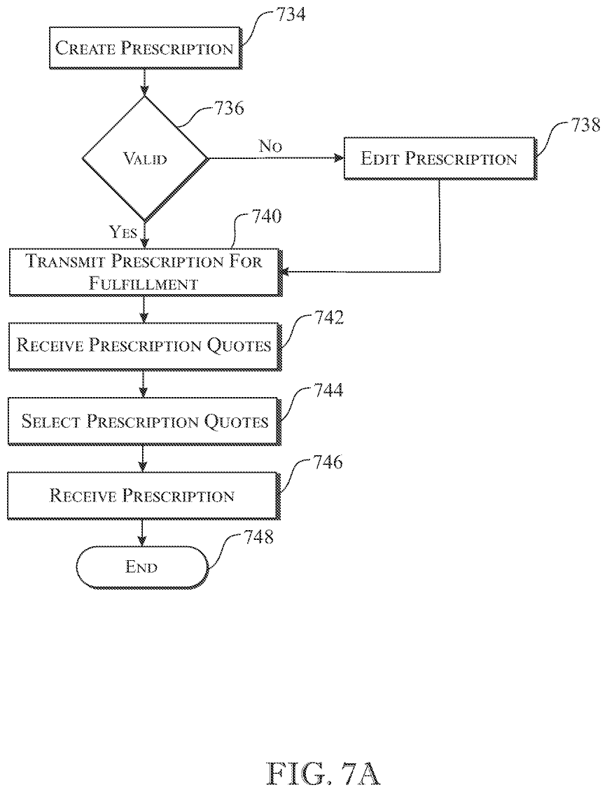

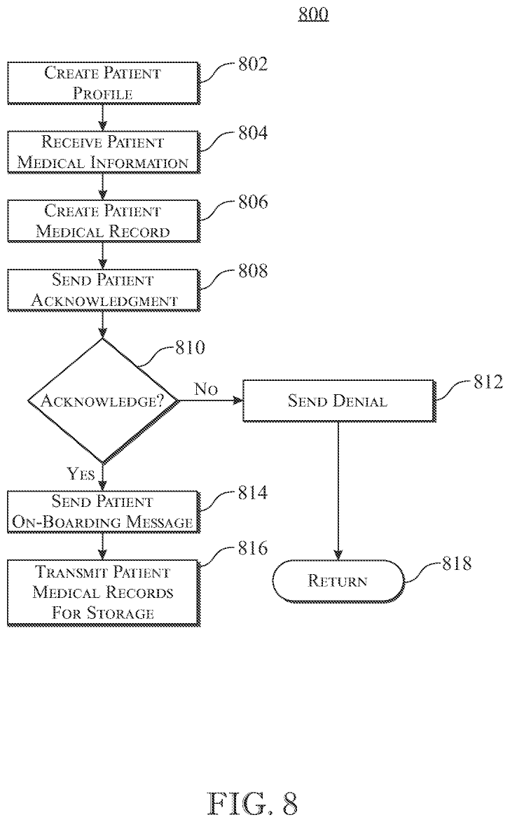

Turning our attention to FIGS. 7 and 7A, a flowchart of illustrative operations for patient control is presented for storing, accessing, managing, securing and sharing their patient medical records using the cloud-based medical record management system of FIG. 1 in accordance with an embodiment. In accordance with the operations of FIG. 7 that begin in step 702, a standard log-in procedure at step 704 is undertaken by the medical professional user (e.g., doctor, nurse, nurse practitioner, etc.) that will attend to the patient seeking medical attention. As will be appreciated, such login procedure may include entering a username and password (and second factor authentication) specific to the medical professional user. After logging in, the medical professional user (associated with, for example, medical facility 116), at step 706, receives a patient medical record access request indicating a patient is seeking some sort of medical treatment. At step 708, a determination is made whether such patient is an existing patient of such medical professional user and if not, at step 710 control is transferred to the patient registration operations for onboarding as shown in FIG. 8.

Turning our attention to FIG. 8, a flowchart of illustrative operations 800 is shown for new patient (e.g., the patient 108) onboarding using the cloud-based medical record management system of FIG. 1 in accordance with an embodiment. Such operations begin with creating a patient profile at step 802 and thereafter receiving, at step 804, the patient medical information from the new patient requesting medical services. At step 806, a patient medical record is created and a communication (e.g., an email, SMS message or any other like communication type) is sent to the patient, at step 808, for acknowledgment. At step 810, a determination is made if the patient has confirmed and acknowledged creating of his medical record and if not the, at step 812, a denial message is sent to the user and the operations return, at step 818, to the calling procedure (i.e., step 708 of FIG. 7).

If a patient acknowledgement is received, a patient on-boarding message is sent to the patient at step 814 indicating they have been accepted as a new patient and, at step 816, their newly created patient medical is transmitted for storage to the database 106 in the HIPAA-compliant cloud 102, as detailed herein above. Having completed the on-boarding of the new patient the operations return, at step 818, to the calling procedure (i.e., step 708 of FIG. 7).

Turning back to FIG. 7, the patient profile is retrieved at step 712 and a determination is made, at step 714, as to whether patient controlled access has been received and granted by that patient. As detailed herein above, the patient 108 employs two primary mechanisms to facilitate such control, in particular, the patient device 110 and the patient control card 112. In an embodiment in which the patient 108 employs the patient device and the patient app 202, illustratively, the patient app 202 will transmit the patient ID 610 (see, FIG. 6) and the access code 612 back to, for example, the medical facility 116 where the patient 108 is seeking medical services. Upon receipt of such patient ID 610 and access code 612, the patient's record is retrieved for viewing, at step 726, by the medical professional which may include a dashboard, reports, drug list and pharmacy list as well as other like pertinent information. At step 728, the patient 108 is added to the queue for examination and the examination is performed, at step 730, by the medical professional in the normal course. Upon examination completion, the medical professional, at step 732, enters the examination results, drugs prescribed and test data.

In accordance with the disclosed embodiments a number of additional operational features may be available to the medical professional (e.g., the doctor) in the execution of the log-in operation (i.e., step 704) referenced herein above and/or the upon the retrieval of the patient profile (i.e., step 712) after receiving the patient medal records profile access request (i.e., step 706). For example, the medical professional may be able view a patient queue of those patient's awaiting examination including, but not limited to, the particular patient that has just been authenticated. Further, the medical professional may be able to view the existing medications and prescriptions associated with an authenticated patient. In the event the medical professional has unsigned prescriptions awaiting execution, these may be viewed and selected for execution. Such viewing may be subject to the entering of unique pin code issued to the medical professional for security purposes. A number of reports may also be available for viewing by the medical professional regarding any number of matters concerning patient care and the administration of medical and prescription services. The medical professional may also be able to view specific consultation fees and their associated details, and the collection status thereof. In the event the medical professional wants to view staffing activities, the medical professional may be able to select a particular staff member and view an associated activity log. Further, if a patient needs their patient card (e.g., as associated with their patient ID 610 and access code 612) re-issued, the medical professional will be able to select the particular patient, complete all the requested details, and a new patient card will be issued to this patient.

In typical cases, a prescription is needed by the patient 108 to obtain the particular prescribed drugs from a pharmacy. As such and as shown in FIG. 7A, at step 734, one or more prescriptions is created and a determination is made, as step 736, whether the prescription is valid. If not, at step 738, the prescription is edited, and approval sought. Once approved, the prescription is transmitted, at step 740 for fulfillment. In accordance with the embodiment, this fulfillment transmission is made to multiple pharmacies anonymously (e.g., pharmacy 124) through the HIPAA-compliant cloud 102 and in this way the patient 108 may receive multiple fulfillment quotes, at step 742, and may select one, at step 744 a smartphone, laptop computer, tablet and/or wearable device, based the patient's personal preferences such as pharmacy location or price, to name just a few preferences. Advantageously, this provides the patient 108 with control and authority over fulfillment in view of the communicated price levels and visibility into the price variations among the competing pharmacies. Once selected, the patient 108 receives, at step 746, the prescription and the operations end at step 748.

As just detailed, the patient 108 employed the patient device 110 to request and grant access to his medical information, for example, an instantiation of the patient medical record 600 as stored in the databases 106. In an alternative embodiment, at step 716 a determination is made whether the patient 108 possesses the patient control card 112 and, if so, the patient control card 112 is swiped at step 722 in a well-known fashion using a smart card reader. At step 722, a determination is made whether the swiped information from the control card 112 allowed for patient verification and, if so, the process returns to step 726, whereby the patient record is retrieved providing the medical professional with information such as a dashboard, reports, drug list and pharmacy list as well as other like pertinent information. If not, the process is transferred to step 802 (see, FIG. 8) for the creation of a patient profile. At step 728, the patient is added to the queue for examination and the examination is performed, at step 730, by the medical professional in the normal course. Upon examination completion, the medical professional, at step 732, enters the examination results, drugs prescribed, test data and other relevant information. Again, in typical cases, as noted above, a prescription is needed by the patient to obtain the particular prescribed drugs from a pharmacy.

As such, at step 734, as shown in FIG. 7A, one or more prescriptions is created and a determination is made, as step 736, whether the prescription is approved. If not, at step 738, the prescription is edited, and approval sought. Once approved, the prescription is transmitted, at step 740 for fulfillment. In accordance with the embodiment, this fulfillment transmission is made to multiple pharmacies (e.g., pharmacy 124) through the HIPAA-compliant cloud 102 and in this way the patient 108 may receive multiple fulfillment quotes, at step 742, and may select one, at step 744, based the patient's personal preferences such as pharmacy location or price, to name just a few preferences. Advantageously, this provides the patient 108 with control and authority over fulfillment in view of the communicated price levels and visibility into the price variations among the competing pharmacies. Once selected, the patient 108 receives, at step 746, the prescription and the operations end at step 748.

Thus, in accordance with the embodiments detailed herein above, the patient 108 is provided complete control over access to his medical records and is provided a transparent system for sharing, securing and transmitting to various medical providers over various medical system platforms. In this way, medical professional, medical facilities and other healthcare-related organizations are able to communicate and share medical records, under patient specific control, in a rapid, secure way and in real-time.

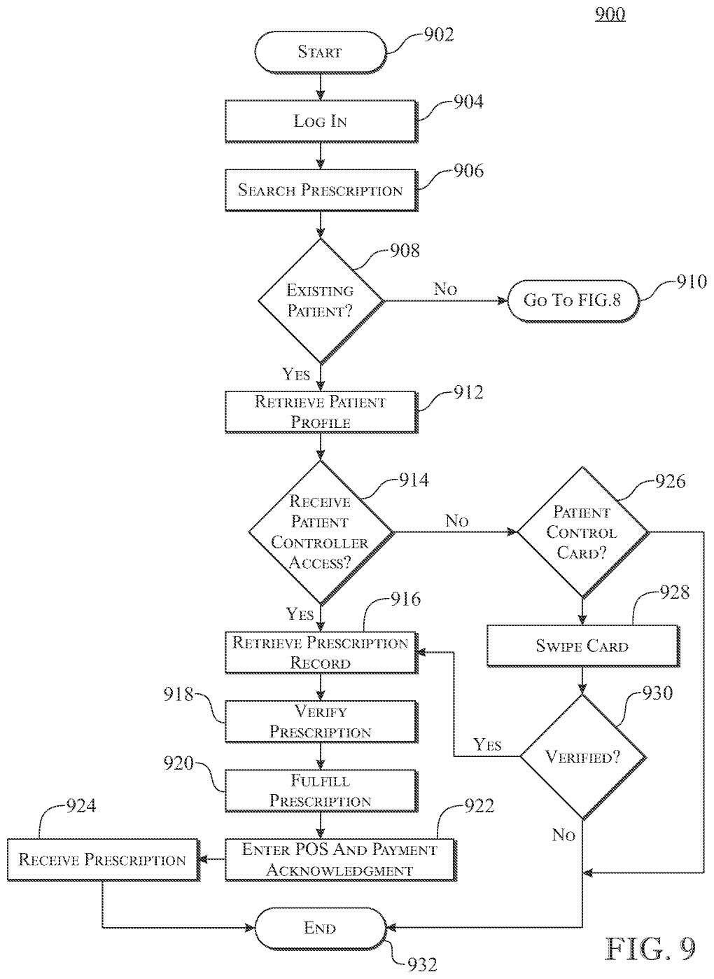

As noted above, one of the aspects of the disclosed embodiments provides significant advantages to the patient 108 in procuring necessary prescription drugs in view of the patient's direct control and authority over fulfillment in view of the communicated price levels and visibility into the price variations among the competing pharmacies. Turning our attention to FIG. 9, a flowchart of illustrative operations 900 is presented for prescription fulfillment using the cloud-based medical record management system of FIG. 1 in accordance with an embodiment. In accordance with the operations 900 of FIG. 9 that begin in step 902, the pharmacist logs in to the system, at step 904, and a search is performed, at step 906, for the patient's (e.g., the patient 108) prescription as received from the HIPAA-compliant cloud 102 as detailed above. In accordance with the embodiment, the patient 108 may be physically present in the same facility as the pharmacist or located in a remote location. At step 908, a determination is made whether such patient is an existing patient (or customer) of the pharmacy and if not, at step 910 control is transferred to the patient registration operations which are the same as those detailed previously in FIG. 8 with the exception that at step 818 of FIG. 8 control returns to the calling function at step 910 in the present embodiment.

Once the status of the patient is established, the patient profile is retrieved at step 912 and a determination is made, at step 914, as to whether patient controlled access has been received and granted by that patient. As detailed herein above, the patient 108 employs two primary mechanisms to facilitate such control, in particular, the patient device 110 and the control card 112. In an embodiment in which the patient 108 employs the patient device 110 and patient app 202, illustratively, the patient app 202 will transmit the patient ID 610 (see, FIG. 6) and the access code 612 (see, FIG. 6) back to, for example, the pharmacy 124 where the patient is seeking to fulfill the prescription. Upon receipt of the patient ID 610 and the access code 612, the patient profile is unlocked for viewing by the pharmacist as well as other like pertinent information, for example, if the patient 108 has known allergies. At step 916, the prescription record is retrieved, and the prescription is verified and/or edited by the pharmacist at step 918 and, at step 920, the prescription is fulfilled in the normal course.

In a further embodiment, when and if the medical professional is a pharmacy owner, he/she may have additional privileges including but not limited to the pharmacist centric operations detailed herein above. Illustratively, the pharmacy owner is akin to a so-called “super administrator” whereby they have additional or different responsibilities. In accordance with an embodiment, a number of additional operational features may be available to the pharmacy owner in the execution of the log-in operation (i.e., step 904) referenced herein above and/or the upon the retrieval of the patient profile (i.e., step 912). Illustratively, these additional operational features may include viewing various dashboards and/or reports regarding the activities of the pharmacy and/or the individual pharmacists. Further, user management functions may be reserved for the pharmacy owner such as onboarding procedures (e.g., see FIG. 8).

As detailed herein above, in accordance with an embodiment, there may have been a fulfillment transmission made to multiple pharmacies (e.g., pharmacy 124) through the HIPAA-compliant cloud 102 and in this way the patient 108 may receive multiple fulfillment quotes which can be compared in real-time by the patient 108 as to whether the just filled prescription is competitive with the multiple fulfillment quotes before proceeding with payment. Upon completion, the pharmacist, at step 922, enters, for example, a point of service (POS) and payment acknowledgement. Once fulfilled and paid for, the patient 108 receives, at step 924, the prescription and the operations end at step 932. Advantageously, this provides the patient 108 with control and authority over fulfillment in view of the prescription.

As just detailed, the patient 108 employed the patient device 110 to fulfill his prescription request using the patient medical record 600 as stored in databases 106. In an alternative embodiment, at step 926 a determination is made whether the patient 108 possesses the patient control card 112 and, if not the operations end at step 932. If so, the patient control card 112 is swiped, at step 928, in a well-known fashion using a smart card reader, for example, by the pharmacist at the pharmacy 124. At step 930, a determination is made whether the swiped information from the control card 112 allowed for patient verification by receipt of the patient ID 610 and the access code 612 and, if not the operations end at step 932. If verified, the prescription record is retrieved for viewing, at step 916, by the pharmacist as well as other like pertinent information, for example, if the patient 108 has any known allergies. At step 918, the prescription is verified and/or edited by the pharmacist and, at step 920, the prescription if filled in the normal course. Upon completion, the pharmacist, at step 922, enters, for example, a point of service (POS) and payment acknowledgement. Once fulfilled and paid for, the patient 108 receives, at step 924, the prescription and the operations end at step 932. Advantageously, this provides the patient 108 with direct control and authority over fulfillment of the prescription.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

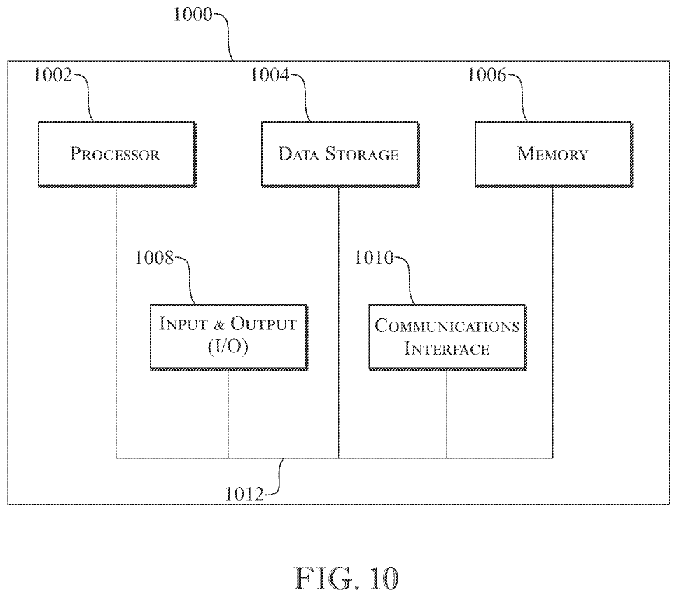

For example, FIG. 10 is a high-level block diagram of an exemplary computer 1000 that may be used for implementing a cloud-based medical record management system and associated methodologies that provide patient control for storing, accessing, managing, securing and sharing their patient medical records in accordance with the various embodiments herein. Computer 1000 comprises a processor 1002 operatively coupled to a data storage device 1004 and a memory 1006. Processor 1002 controls the overall operation of computer 1000 by executing computer program instructions that define such operations. Communications bus 1012 facilitates the coupling and communication between the various components of computer 1000. The computer program instructions may be stored in data storage device 1004, or a non-transitory computer readable medium, and loaded into memory 1006 when execution of the computer program instructions is desired.

Thus, the steps of the disclosed method (see, e.g., FIGS. 7-9) and the associated discussion herein above can be defined by the computer program instructions stored in memory 1006 and/or data storage device 1004 and controlled by processor 1002 executing the computer program instructions. For example, the computer program instructions can be implemented as computer executable code programmed by one skilled in the art to perform the illustrative operations defined by the disclosed methods. Further, it will be appreciated that any flowcharts, flow diagrams, state transition diagrams, pseudo code, program code and the like represent various processes which may be substantially represented in computer readable medium and so executed by a computer, machine or processor, whether or not such computer, machine or processor is explicitly shown. One skilled in the art will recognize that an implementation of an actual computer or computer system may have other structures and may contain other components as well, and that a high level representation of some of the components of such a computer is for illustrative purposes.

Accordingly, by executing the computer program instructions, processor 1002 executes an algorithm defined by the disclosed method. Computer 1000 also includes one or more communications interface 1010 for communicating with other devices via a network (e.g., a wireless communications network) or communications protocol (e.g., Bluetooth®). For example, such communication interfaces may be a receiver, transceiver or modem for exchanging wired or wireless communications in any number of well-known fashions. Computer 1000 also includes one or more input/output devices 1008 that enable user interaction with computer 1000 (e.g., camera, display, keyboard, mouse, speakers, microphone, buttons, etc.).

Processor 1002 may include both general and special purpose microprocessors and may be the sole processor or one of multiple processors of computer 1000. Processor 1002 may comprise one or more central processing units (CPUs), for example. Processor 1002, data storage device 1004, and/or memory 1006 may include, be supplemented by, or incorporated in, one or more application-specific integrated circuits (ASICs) and/or one or more field programmable gate arrays (FPGAs).

Data storage device 1004 and memory 1006 each comprise a tangible non-transitory computer readable storage medium. Data storage device 1004, and memory 1006, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1008 may include peripherals, such as a camera, printer, scanner, display screen, etc. For example, input/output devices 1008 may include a display device such as a cathode ray tube (CRT), plasma or liquid crystal display (LCD) monitor for displaying information to the user, a keyboard, and a pointing device such as a mouse or a trackball by which the user can provide input to computer 1000.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A medical record management system comprising:
a healthcare information exchange comprising:
at least one database for storing a plurality of patient specific medical records;
a processor;
a memory storing instructions that when executed cause the processor to perform operations comprising:
receiving, in real-time, a patient medical record access request specific to and controlled solely by a patient, the patient medical access request including at least a patient identification code and an access code that are specific to identifying and authenticating the patient;
verifying, in response to receiving the patient medical record access request an authenticity of the patient using the patient identification code and the access code and, only if the authenticity is verified, retrieving in real-time, from the at least one database, a particular one patient specific medical record of the plurality of patient specific medical records, the particular one patient specific medical record being associated with the patient; and
transmitting, in real-time, the particular one patient specific medical record to at least one healthcare entity of the plurality of healthcare entities.

2. The medical record management system of claim 1, wherein the verifying the authenticity of the patient operation further comprises:
routing, in real-time, the patient medical record access request to a medical professional associated with the at least one healthcare entity indicating the patient is seeking medical treatment.

3. The medical record management system of claim 2, wherein the verifying the authenticity of the patient operation further comprises:
determining, by and through the medical professional and in real-time, whether the patient is an existing patient of the medical professional.

4. The medical record management system of claim 3, wherein the operations further comprise:
retrieving, but only if the patient is determined to be the existing patient of the medical professional, a patient profile specific to the patient.

5. The medical record management system of claim 1, wherein the patient medical record access request specific to and controlled solely by the patient is transmitted from a user device associated with the patient.

6. The medical record management system of claim 5, wherein the user device is one of a smartphone, a laptop computer, a tablet or a wearable device.

7. The medical record management system of claim 4, wherein the operations further comprise:
fulfilling, using the patient profile retrieved and in real-time, at least one prescription as prescribed by the medical professional for the patient.

8. A medical record management system comprising:
a healthcare information exchange comprising:
at least one database for storing a plurality of patient specific medical records;
a processor;
a memory storing instructions that when executed cause the processor to perform operations comprising:
receiving, in real-time, a patient medical record access request specific to and controlled solely by a patient, the patient medical access request triggered by the patient using a patient control card, the patient control card comprising at least a patient identification code and an access code that are specific to identifying and authenticating the patient;
verifying, in response to receiving the patient medical record access request an authenticity of the patient using the patient identification code and the access code and, only if the authenticity is verified, retrieving in real-time, from the at least one database, a particular one patient specific medical record of the plurality of patient specific medical records, the particular one patient specific medical record being associated with the patient; and
transmitting, in real-time, the particular one patient specific medical record to at least one healthcare entity of the plurality of healthcare entities.

9. The medical record management system of claim 8, wherein the verifying the authenticity of the patient operation further comprises:
routing, in real-time, the patient medical record access request to a medical professional associated with the at least one healthcare entity indicating the patient is seeking medical treatment.

10. The medical record management system of claim 9, wherein the verifying the authenticity of the patient operation further comprises:
determining, by and through the medical professional and in real-time, whether the patient is an existing patient of the medical professional.

11. The medical record management system of claim 10, wherein the operations further comprise:
retrieving, but only if the patient is determined to be the existing patient of the medical professional user, a patient profile specific to the patient.

12. The medical record management system of claim 11, wherein the operations further comprise:
transmitting, in real-time, a plurality of prescription fulfillment quotes generated as a function of at least one prescription as prescribed the medical professional for the patient to a user device associated with the patient.

13. The medical record management system of claim 12, wherein the operations further comprising:
fulfilling, using the patient profile retrieved and in real-time, the at least one prescription from a particular one of the prescription fulfillment quotes.

14. The medical record management system of claim 10, wherein the operations further comprise:
adding, but only if the patient is determined to be the existing patient of the medical professional user and in real-time, a name of the patient to an examination queue.

15. A non-transitory computer-readable medium storing computer program instructions for executing medical records management, which, when executed on a processor, cause the processor to perform operations comprising:
receiving, in real-time, a patient medical record access request specific to and controlled solely by a patient, the patient medical access request including at least a patient identification code and an access code that are specific to identifying and authenticating the patient;
verifying, in response to receiving the patient medical record access request an authenticity of the patient using the patient identification code and the access code and, only if the authenticity is verified, retrieving in real-time, from at least one database, a particular one patient specific medical record of the plurality of patient specific medical records, the particular one patient specific medical record being associated with the patient; and
transmitting, in real-time, the particular one patient specific medical record to at least one healthcare entity of the plurality of healthcare entities.

16. The non-transitory computer-readable medium of claim 15, wherein the verifying the authenticity of the patient operation further comprises:
routing, in real-time, the patient medical record access request to a medical professional associated with the at least one healthcare entity indicating the patient is seeking medical treatment.

17. The non-transitory computer-readable medium of claim 16, wherein the verifying the authenticity of the patient operation on further comprises:
determining, by and through the medical professional and in real-time, whether the patient is an existing patient of the medical professional.

18. The non-transitory computer-readable medium of claim 17, wherein the operations further comprise:
retrieving, but only if the patient is determined to be the existing patient of the medical professional and in real-time, a patient profile specific to the patient.

19. The non-transitory computer-readable medium of claim 15, wherein the patient medical record access request specific to and controlled solely by the patient is transmitted from a user device associated with the patient.

20. The non-transitory computer-readable medium of claim 18, wherein the operations further comprise:
transmitting, in real-time, a plurality of prescription fulfillment quotes generated as a function of at least one prescription as prescribed by the medical professional for the patient, to a user device associated with the patient; and
fulfilling, using the patient profile retrieved, the at least one prescription from a particular one of the prescription fulfillment quotes.
